(19) United States
(12) Patent Application Publication
(10) Pub. No.: US 2003/0104417 A1
(43) Pub. Date: Jun. 5, 2003

(54) Template-mediated, ligation-oriented method of nonrandomly shuffling polynucleotides

(75) Inventors: Daniel Dupret, Calvisson (FR); Jean Michel Masson, Toulouse (FR); Fabrice Lefevre, Nimes (FR)

Correspondence Address: HUNTON & WILLIAMS, INTELLECTUAL PROPERTY DEPARTMENT, 1900 K STREET, N.W., SUITE 1200, WASHINGTON, DC, 20006-1109, US

(73) Assignee: Proteus S.A., Nimes (FR)

(21) Appl. No.: 10/131,175

(22) Filed: Apr. 25, 2002

Related U.S. Application Data
(63) Continuation-in-part of application No. 09/723,316, filed on Nov. 28, 2000.
(63) Continuation-in-part of application No. 09/840,861, filed on Apr. 25, 2001.
(60) Provisional application No. 60/285,998, filed on Apr. 25, 2001.

(30) Foreign Application Priority Data
Aug. 12, 1998 (FR) 98/10338
Aug. 11, 1999 (WO) PCT/FR99/01973
Apr. 25, 2002 (WO) PCT/FR02/01435

(51) Int. Cl. C12Q 1/68; C12P 19/34; C12M 1/34

(52) U.S. Cl. 435/6; 435/287.2; 435/91.2

(57) ABSTRACT

Method of gene shuffling using oriented ligation, whereby at least two fragments are adjacently hybridized on an assembly template. Invention is particularly aimed at generating novel polynucleotides that differ in some advantageous respect compared to a reference sequence. Invention further includes sequences created by the method, hosts and vectors containing same, and proteins translated therefrom.

### CROSS-REFERENCE TO RELATED APPLICATIONS

This application claims benefit of priority of the following applications: U.S. application Ser. No. 09/840,861, filed Apr. 25, 2001; U.S. Provisional Application No. 60/285,998, filed Apr. 25, 2001; U.S. application Ser. No. 09/723,316, filed Nov. 28, 2000; PCT Application No. PCT/FR99/01973, filed Dec. 8, 1999; French Patent Application No. FR98/10338, filed Dec. 8, 1998; and the closest foreign equivalent to the instant application, a PCT Application filed by the instant Applicant on Apr. 25, 2002. The foregoing applications are herein incorporated by reference in their entireties.

### BACKGROUND OF THE INVENTION

The present invention relates broadly to genetic recombination and to the field known variously as directed evolution, molecular breeding or DNA shuffling. The invention aims particularly at generating novel sequences with improved characteristics compared to those of a reference sequence. When performed outside a living organism, the process comprises a technique for in vitro evolution. The invention further relates to the sequences generated by the method, libraries of such sequences, hosts and vectors containing such sequences, proteins translated therefrom, to arrays that simulate the method of the invention, and to arrays in which the method can be performed. The invention further relates to intermediate products of the method, to reaction mixtures of certain types of polynucleotide fragments and assembly templates, and to compositions of certain assembly templates and recombinant polynucleotides produced therewith.

Various techniques are known to facilitate in vitro recombination of polynucleotide sequences. The most well-known conventional techniques are DNA shuffling with sexual PCR (multiple cycles with no added primer) and staggered extension (StEP), which both rely on polymerization.

Typically, in DNA shuffling with sexual PCR, DNase I randomly cuts polynucleotide sequences to form oligonucleotide fragments, the fragments initiate polymerization or PCR extension, and the recombined polynucleotides are amplified. At each hybridization step, crossovers occur at homologous regions among the sequences (“strand switching”). A schematic representation of this method appears in FIG. 1A.

StEP consists of mixing various polynucleotide sequences containing various mutations in the presence of a pair of initiators. This mixture is subjected to PCR reactions in which the hybridization and polymerization steps are consolidated into a single, very brief step. These conditions make it possible to hybridize the initiators but also slow the polymerization so that the initiators have time to synthesize only fragments which, after denaturation, re-hybridize randomly to the various polynucleotide sequences. A schematic representation of this method appears in FIG. 1B.

Relying heavily on polymerization has drawbacks. Such methods do not confer control over the rate or location of recombination, which occurs randomly during the successive stages of polymerization. Depending on the conditions and polymerase used, the polymerization can also produce either undesired supplemental mutations or insufficient numbers of mutations. The latter occurs when long gaps are filled with residues that are fully complementary to the opposite strand. Further, after enough cycles, the fragments grow very long and become what are known as “mega-initiators” (6). Mega-initiators can cause various problems, particularly when the starting polynucleotides exceed about 1.5 kb.

### ADVANTAGES OF THE INVENTION

The invention need not rely on polymerization, size fractionation (isolation of fragments by size) or amplification of the initial polynucleotides or fragments. Further, Applicant believes, though not wishing except where stated otherwise to be limited thereto in any way, that the invention and embodiments confer broad advantages.

First, the invention provides control over the locations of recombination. Hybridization on a template enables precise control of the locations where recombination occurs. For example, if a target protein contains an active site that one desires to leave unchanged, the invention is capable of limiting recombination to regions other than the active site. Furthermore, the invention can achieve high recombination between closely neighboring sequence segments. Rather than treating close-lying sequences as “linked,” and moving them in chunks, the invention can separate the close-lying sequences. Therefore, in a sense the invention also achieves high resolution, fidelity and quality of genetic diversity. Indeed, the embodiment of the invention that employs nonrandom fragmenting can use fragments as short as 15 residues.

The invention may also generate more recombination and incorporation of fragments per reaction cycle, particularly in embodiments other than ligation-only embodiments (defined below). In other words, it achieves a high quantity of genetic diversity. High quantity is achieved directly by stimulating more total recombination events. It is achieved indirectly by increasing overall efficiency. Overall efficiency is increased by using, inter alia, oriented ligation. Without oriented ligation, a sequence cut into “n” fragments will reassociate into an enormous variety of possible forms, even if only one or a few forms are useful. The present invention, on the other hand, facilitates direct achievement of the desired form. Indeed, in some embodiments of the invention, it is possible to obtain a recombinant polynucleotide after only a single reaction cycle.

Typically, the invention further increases efficiency by generating relatively few unshuffled parental clones and duplicate chimeras. Avoiding these unwanted by-products provides room for more novel chimeras. The conventional methods may produce screening libraries that consist of 30% to 70% parental DNA. In all methods of directed evolution, molecular breeding or gene shuffling, a screening library of recombinant DNA molecules is produced and these molecules are expressed and screened. Screening is the most expensive and time-consuming part of the process since the libraries may contain 100,000 to several million recombinant molecules. Eliminating parental DNA from the screening libraries mitigates this problem. The elimination of parental DNA is enhanced when the template is transient, as in more preferred embodiments of the invention, because the final population is composed of only the new, variant polynucleotides.

Preferred embodiments of the method, particularly those that employ solitary-stranded templates or fragments, also facilitate low-homology shuffling, e.g., of distantly-related members of gene families. The term “solitary-stranded” is used to describe a population of particular single-stranded sequences that do not complement each other because they are all from the same strand, either the sense or antisense strand, of one polynucleotide or multiple homologous polynucleotides. Since solitary-stranded fragments, for example, are not complementary or at least not strictly complementary to another fragment in the reaction mixture, hybridization is not biased toward the “wild type” sequences that would be formed by complementary fragments. Hybridization temperatures can be adjusted to the degree of homology among the sequences, thereby maximizing diversity and greatly increasing the chances of finding the right mutant in the shortest number of recombination cycles. (Note that the invention may still comprise achieving a desired bias, e.g., by using higher amounts of one parental polynucleotide.)

In addition, the invention demands little preparation of the starting DNA library. The invention allows immediate use of complex or genomic DNA which may include introns. Some other methods require time-consuming isolation of mRNA and re-creation of the cDNA sequence in order to generate fragments for shuffling or reassembly.

Additional advantages of the invention or its embodiments are further described herein.

### SUMMARY OF THE INVENTION

Although the present invention relates broadly to genetic recombination, “recombination” is somewhat of a misnomer with regard to the invention insofar as the term implies that two strands disassociate and then recombine with each other to form a recombinant sequence. In other words, the invention does not rely on strand switching or crossovers. Nevertheless, “recombination” and related terms are retained herein, subject to this caveat.

In one embodiment, the method of the invention includes:

A template-mediated, ligation-oriented method for nonrandomly shuffling polynucleotides, comprising:

a) obtaining, directly or indirectly from a polynucleotide library, single-stranded fragments of at least two homologous polynucleotides;

b) hybridizing said fragments to one or more devised assembly templates until at least two of the fragments are adjacently hybridized, thereby forming at least one partially double-stranded polynucleotide, wherein at least one of said templates shares at least one zone of homology with said homologous polynucleotides;

c) treating said partially double-stranded polynucleotide to form at least one recombinant polynucleotide,

wherein said treating comprises, in any order, the following:

(i) ligating nicks, and

(ii) where necessary, any one of or any combination of the following gap filling techniques:

filling in gaps by further hybridizing said fragments to said templates to increase the number of fragments that are adjacently hybridized,

filling in short gaps by trimming any overhanging flaps of any partially hybridized fragments, and

filling in short gaps via polymerization.

In the above embodiment, any of the steps may be repeated as necessary, particularly steps (b) and (c). In another embodiment, the method of the invention generates a recombinant polynucleotide after only one round, cycle or single operation of each step of the invention. In a preferred embodiment, the method further comprises step (d) selecting at least one of said recombinant polynucleotides that has a desired property. More preferably, the steps occur in vitro (outside a living organism). In some preferred embodiments, the method employs, inter alia, nonrandom fragmentation, transient templates, and solitary-stranded templates or fragments.

In an alternative embodiment, the invention essentially comprises steps (b) and (c) above. In such case, step (b) becomes “step (a)” and also includes hybridizing single stranded fragments of at least two homologous polynucleotides.”

In another alternative embodiment, the invention comprises a template-mediated, ligation-oriented method for nonrandom low-homology shuffling of gene families in vitro. Whether homology is considered low differs in different contexts, but homology that ranges below 50% (e.g., 40-70% or 20-45%) would typically be considered low. In another alternative embodiment, the parental polynucleotides vary in length by more than two residues.

In yet another alternative embodiment, the invention comprises a template-mediated, ligation-oriented method for in vitro nonrandom shuffling of mutation-containing zones of polynucleotide alleles. This embodiment further comprises locating restriction sites for mutation-containing zones among the alleles, and obtaining fragments corresponding to those restriction sites.

The invention further includes sequences created by the method, libraries of same, hosts and vectors containing same and proteins translated therefrom. It also includes a logical array, such as a computer algorithm, that simulates the inventive method, or a physical array, such as a biochip, in which the inventive method may be performed. The invention further relates to intermediate products of the method, to reaction mixtures of polynucleotide fragments and assembly templates that can be used to carry out some or all steps of the method, and to compositions of certain assembly templates and recombinant polynucleotides produced therewith.

### DEFINITIONS

“In vitro”, as used herein, refers to any location outside a living organism.

“Homologous” polynucleotides differ from each other at least at one corresponding residue position. Thus, as used herein, “homologous” encompasses what is sometimes referred to as “partially heterologous.” The homology, e.g., among the parental polynucleotides, may range from 20 to 99.99%, preferably 30 to 90, more preferably 40 to 80%. In some embodiments the term homologous may describe sequences that are, for example, only about 20-45% identical at corresponding residue positions. Homologous sequences may or may not share with each other a common ancestry or evolutionary origin.

“Polynucleotide” and “polynucleotide sequence” refer to any nucleic or ribonucleic acid sequence, including mRNA, that is single-stranded, solitary-stranded or partially or fully double-stranded. When partially or fully double-stranded, each strand may be identical or heterologous to the other, unless indicated otherwise. A polynucleotide may be a gene or a portion of a gene. “Gene” refers to a polynucleotide or portion thereof associated with a known or unknown biological function or activity. A gene can be obtained in different ways, including extraction from a nucleic acid source, chemical synthesis and synthesis by polymerization. “Parental polynucleotide” and “parent” are interchangeable synonyms that refer to the polynucleotides that are fragmented to create donor fragments. Parental polynucleotides are often derived from genes. “Recombined polynucleotide,” “mutant polynucleotide,” “chimeric polynucleotide” and “chimera” generally refer to the polynucleotides that are generated by the method. However, these terms may refer to other chimeric polynucleotides, such as chimeric polynucleotides in the initial library. “Reference sequence” refers to a polynucleotide, often from a gene, having desired properties or properties close to those desired, and which is used as a target or benchmark for creating or evaluating other polynucleotides.

“Polynucleotide library” and “DNA library” refer to a group, pool or bank of polynucleotides containing at least two homologous polynucleotides or fragments thereof. A polynucleotide library may comprise either an initial library or a screening library. “Initial library,” “initial polynucleotide library,” “initial DNA library,” “parental library” and “start library” refer to a group, pool or bank of polynucleotides or fragments thereof containing at least two homologous parental polynucleotides or fragments thereof. The initial library may comprise genomic or complex DNA and include introns. It may also comprise sequences generated by prior rounds of shuffling. Similarly, a screening library or other limited library of recombinant polynucleotides or fragments may serve as and be referred to as an initial library. “Screening library” refers to the polynucleotide library that contains chimeras generated by the inventive process or another recombinant process.

“Residue” refers to an individual nucleotide or ribonucleotide, rather than to multiple nucleotides or ribonucleotides. Residue may refer to a free residue that is not part of a polynucleotide or fragment, or to a single residue that forms a part of a polynucleotide or fragment.

“Donor fragments” and “fragments” generally refer to the fragmented portions of parental polynucleotides. Fragments may also refer to supplemental or substitute fragments that are added to the reaction mixture and/or that derive from a source other than fragmentation of the parental polynucleotides. Most or all of the fragments should be shorter than the parental polynucleotides. In a preferred embodiment, most or all of the fragments are shorter than the assembly templates.

“Nonrandom” and “controlled,” as used herein, refer broadly to the control or predictability, e.g., over the rate or location of recombination, achieved via the template and/or ligation-orientation of the invention. Nonrandom and controlled may also refer more specifically to techniques of fragmenting polynucleotides that enable some control or predictability over the size or sequence of the resulting fragments. For example, using restriction enzymes to cut the polynucleotides provides some control over the characteristics of the fragments. Note that the invention may still be considered nonrandom when it employs random fragmentation (typically by DNase I digestion). In such cases, the assembly template and other features of the invention still provide a degree of control. In preferred embodiments, however, the fragmentation is nonrandom or controlled.

“Assembly template,” “devised template” and “template” refer to a polynucleotide used as a scaffold upon which fragments may anneal or hybridize to form a partially or fully double-stranded polynucleotide. In a preferred embodiment, the template is longer than most or all of the donor fragments. In such a case, the free donor fragments cannot be considered templates for each other. The template may derive from the reference sequence, the initial library, the screening library or elsewhere. Although the template may comprise or derive from a parental polynucleotide of the initial library, a polynucleotide does not qualify as a template if it enters the shuffling process accidentally, e.g., by somehow slipping into the hybridization step without being fragmented. In other words, the template is not entirely random or accidental. Rather, at least to some extent it is devised: the template is directly or indirectly obtained for use as a template by a human being, or a computer operated thereby, via purposeful planning, conception, formulation, creation, derivation and/or selection of either a specific desired polynucleotide sequence(s) or a sequence(s) from a source(s) that is likely to contain a desired sequence(s). The template may be synthetic, result from shuffling or other artificial processes, or it may exist in nature. “Transient template” refers to a template that is not itself incorporated into the final recombinant polynucleotides. This transience is caused by separation or disintegration of the template strand of the nonfinal recombinant polynucleotide generated during the method.

“Solitary-stranded” is used to describe a population of single-stranded sequences that do not complement each other because they are all from the same strand, either sense or antisense, of one polynucleotide or multiple homologous polynucleotides. In other words, sequences from the opposing complementary strands are absent, so the population contains no sequences that are complementary to each other. For example, the population of solitary-stranded fragments may consist of fragments of the top strands of the parental polynucleotides, whereas the population of solitary-stranded templates may consist of bottom strands of one or more of the parental polynucleotides.

“Ligation” refers to creation of a phosphodiester bond between two residues.

“Nick” refers to the absence of a phosphodiester bond between two residues that are hybridized to the same strand of a polynucleotide. Nick includes the absence of phosphodiester bonds caused by DNases or other enzymes, as well as the absences of bonds between adjacently hybridized fragments that have simply not been ligated. As used herein, nick does not encompass residue gaps.

“Gap” and “residue gap,” as used herein, refer to the absence of one or more residues on a strand of a partially double-stranded polynucleotide. In some embodiments of the invention, short gaps (less than approximately 15-50 residues) are filled in by polymerases and/or flap trimming. Long gaps are conventionally filled in by polymerases. In the present invention, long gaps may only be filled via hybridization or trimming.

“Hybridization” has its common meaning except that it may encompass any necessary cycles of denaturing and re-hybridization.

“Adjacent fragments” refer to hybridized fragments whose ends are flush against each other and separated only by nicks, not by gaps.

“Ligation-only” refers to embodiments of the invention that do not utilize or require any gap filling, polymerase extension or flap trimming. In ligation-only embodiments, all of the fragments hybridize adjacently. Note that embodiments that are not ligation-only embodiments still use ligation.

“Ligation-oriented” and “oriented ligation” generally represent or refer to a template-mediated process that enables ligation of fragments or residues in a relatively set or relatively predictable order. All embodiments of the invention are ligation-oriented. For example, a ligation-only embodiment is still ligation-oriented.

### BRIEF DESCRIPTION OF THE DRAWINGS

Reference is made to the appended drawings in which:

FIG. 1 is a schematic representation of conventional DNA-shuffling (FIG. 1A) and StEP (FIG. 1B).

FIG. 2 is a schematic representation of an embodiment of the process of the invention and of certain of its variations and applications.

FIG. 3 represents the positions of the ten zones of mutations (Pvu II and Pst I) carried by each mutant of the ponB gene.

FIG. 4 represents the position of the primers used compared to the sequence of the ponB gene.

FIG. 5 represents the migration on agarose gel of RLR and of PCR reaction products of these RLR reactions.

FIG. 6 represents the position of the mutations compared to the restriction fragments.

FIG. 7 depicts the results of error-prone PCR on WT XynA gene using 1% agarose gel.

FIG. 8 depicts thermal inactivation of mutant 33 at 82° C.

FIG. 9 depicts the results of fragmentation of PCR products with six restriction endonucleases, using 3% agarose gel.

FIG. 10 depicts the results of L-Shuffling™ experiments using 1% agarose gel.

FIG. 11 depicts the results of using PCR Pfu on L-Shuffling™ products, using 1% agarose gel.

FIG. 12 depicts thermal inactivation of mutants at 95° C.

FIG. 13 depicts the results of DNaseI fragmentation of Thermotoga neapolitana (A) and Acidobacterium capsulatum (B) genes, using 1% agarose gel.

FIG. 14 depicts the results of L-Shuffling™ experiments, using 1% agarose gel.

FIG. 15A depicts the results of L-Shuffling™ using n cycles of steps (b) and (c), and FIG. 15B shows the PCR amplification of the corresponding L-Shuffling™ products.

FIG. 16 depicts the results of L-Shuffling™ experiments using increased quantities of fragments.

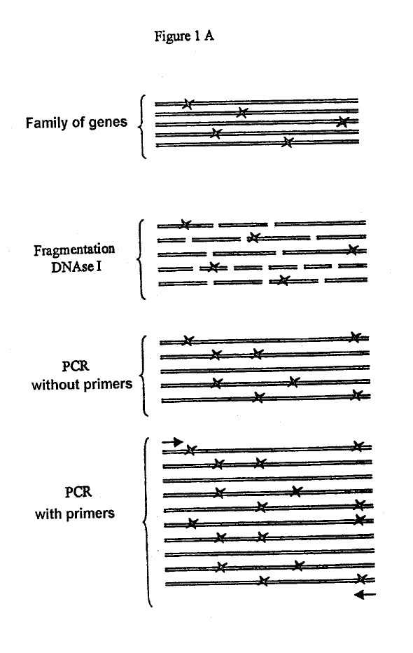

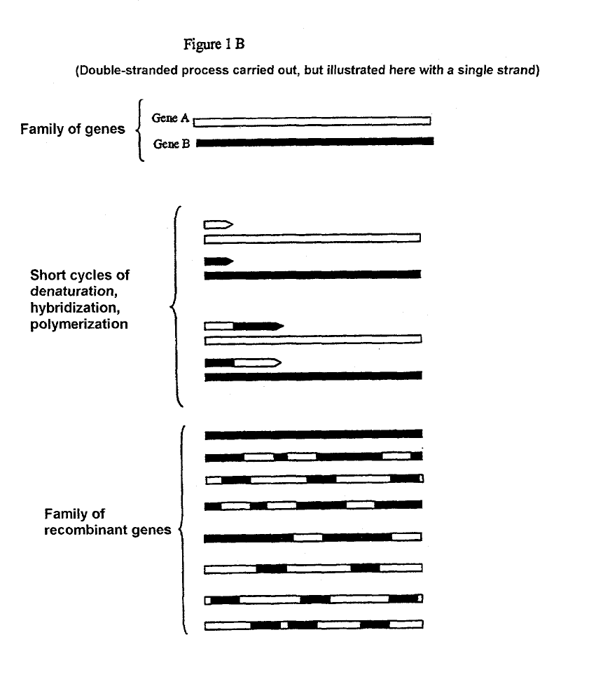

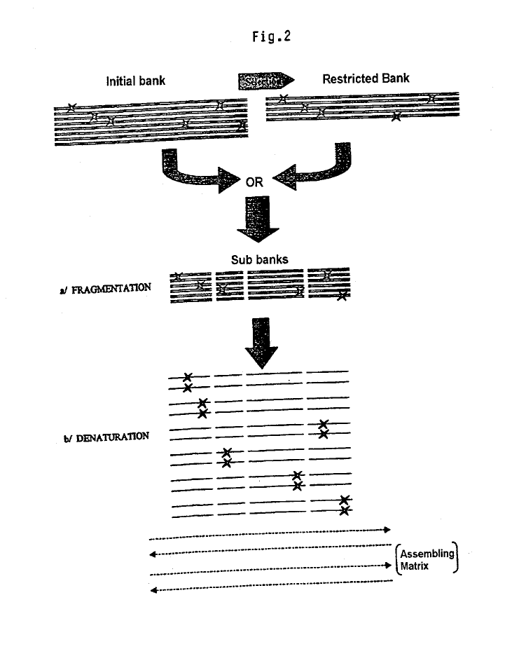

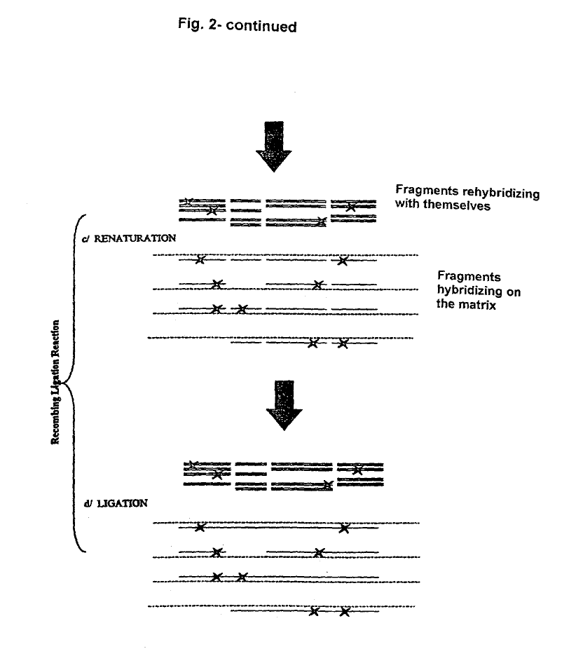

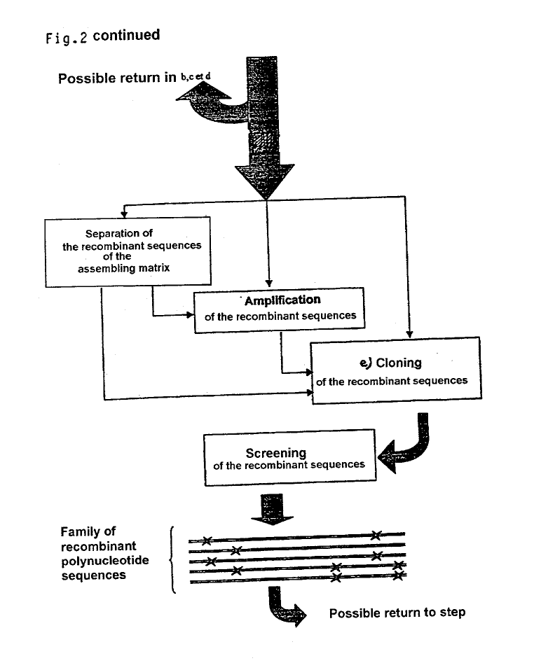

### DETAILED DESCRIPTION OF PREFERRED EMBODIMENTS OF THE INVENTION

One embodiment of the invention comprises a template-mediated, ligation-oriented method for shuffling polynucleotides nonrandomly, comprising:

a) obtaining, directly or indirectly from a polynucleotide library, single-stranded fragments of at least two homologous polynucleotides;

b) hybridizing said fragments to one or more devised assembly templates until at least two of the fragments are adjacently hybridized, thereby forming at least one partially double-stranded polynucleotide, wherein at least one of said templates shares at least one zone of homology with said homologous polynucleotides;

c) treating said partially double-stranded polynucleotide to form at least one recombinant polynucleotide,

wherein said treating comprises, in any order, the following:

(i) ligating nicks, and

(ii) where necessary, any one of or any combination of the following gap filling techniques:

filling in gaps by further hybridizing said fragments to said templates to increase the number of fragments that are adjacently hybridized,

filling in short gaps by trimming any overhanging flaps of any partially hybridized fragments, and

filling in short gaps via polymerization.

Although embodiments of the invention may employ polymerase, such embodiments use polymerase to fill only short gaps (e.g., less than 15-50 residues), not long gaps. In a preferred embodiment, the process employs no polymerase. In ligation-only embodiments, the method employs no gap filling techniques and instead relies on ligation of perfectly adjacent fragments, often achieved after multiple hybridization events.

Preferably, once the partially double-stranded polynucleotides become adequately double-stranded, they are (d) selected for advantageous properties compared to those of one or several reference sequences. Advantageous characteristics may include, for example, thermostability of an enzyme or its activity under certain pH or salinity conditions. Among many other possible uses, such enzymes may be used for desizing textile fibers, bleaching paper pulps, producing flavors in dairy products, or biocatalyzing synthesis of new therapeutic molecules.

The process may also comprise disintegrating the template strand or separating it from the recombinant strand before or after the selection. It may further comprise amplifying the recombinant sequences before selection at step (d), or cloning of recombinant polynucleotide sequences after separation of the recombinant strand from the template. Any amplification technique is acceptable. Due to initiators that can hybridize only to the ends of recombinant sequences, PCR enables selective amplification of the recombinant sequences. However, unlike shuffling with sexual PCR, the invention does not require amplification during the recombination reactions.

A preferred screening techniques entails in vitro expression via in vitro transcription of recombinant polynucleotides, followed by in vitro translation of the mRNAs. This technique eliminates cellular physiological problems and the drawbacks connected with in vivo expression cloning. Further, this technique is easily automated, which enables screening of a high number of recombinant sequences.

Although embodiments of the invention may not in fact need to cycle through any steps more than once (“non-iterative”), the invention also encompasses repetition of any of its steps, such as repetition of steps (a), (b) and/or (c). For instance, the process may or may not entail multiple hybridization events. The hybridization in step (b) and the further hybridization of step (c) are meant to encompass any necessary cycles of denaturing and re-hybridizing. If necessary, the repetition of steps (b) and/or (c) may be performed in part or in whole on ligated and/or non-ligated fragments produced by steps (b) and/or (c), rather than only on the donor fragments produced by step (a). The ligation-only embodiments typically require multiple iterations. In addition to encompassing repetition of steps, the invention includes embodiments that allow simultaneous operation of those steps that are known in the art as capable of simultaneous operation.

In a preferred embodiment, the initial library is itself produced by the present invention. Either in vivo or in vitro screens can be used to form this library for repeating the process of the invention. The recombinant sequences selected after a first running of the process can be optionally mixed with other sequences.

The initial library can also be produced by any method known to one skilled in the art, for example, by starting from a wild-type gene, by successive managed stages of mutagenesis, by “error-prone” PCR (2), by random chemical mutagenesis, by random mutagenesis in vivo, or by combining genes of close or relatively distant families within the same or different species. Preferably, the initial library results from chain polymerization reactions under conditions that create random, localized mutations. The invention may also comprise synthetic sequences.

Assembly Templates

The assembly template of step (b) or (c) is, for example, a polynucleotide from the initial library or a polynucleotide produced therefrom. The template may be synthetic, result from shuffling or other artificial processes, or it may exist in nature. The template can be single- or double-stranded. If double-stranded, it must be denatured, such as in step (b), before actual hybridization can occur. If the template is incorporated directly at step (b), the template must be denatured or already in single-stranded form.

Preferred embodiments use a solitary-stranded template. More preferred embodiments use as a solitary-stranded template the bottom-strand from one parent polynucleotide and top-strand fragments from other parents. This prevents re-annealing of sequences to their own complementary strands. To obtain solitary-stranded DNA molecules, a Bluescript phagemide or a vector of the family of filamentous phages such as M13mp18 can be used. Another method consists in creating double-stranded molecules by PCR by using an initiator phosphorylated at 5′ and the other non-phosphorylated. The digestion of the lambda phage by the exonuclease will destroy the strands of DNA phosphorylated at 5, leaving the non-phosphorylated strands intact. Another method of creating solitary-stranded molecules consists in making an amplification, by asymmetric PCR, starting from a methylated DNA template. Digestion by Dpn I will destroy the methylated strands, leaving intact the amplification products that will then be able to be purified after denaturation.

Preferred embodiments also use transient templates that are not incorporated within the final recombined polynucleotide, e.g., not part of the polynucleotide that is transferred to the screening library. One technique of conferring transience employs markers on either the recombinant strand or the template. For example, the template may be marked by a hapten and separated by, for example, fixing an antihapten antibody on a carrier or by initiating a biotin-streptavidin reaction. Another technique comprises synthesizing a transient template by PCR amplification using methylated dATP, which enables degradation of the template by restriction endonuclease Dpn I. In this case, the recombinant strand must not contain methylated dATP. A transient template can also by prepared by PCR amplification with dUTP, which enables degradation with uracil-DNA-glycosylase. Conversely, it is possible to protect the recombinant strand by amplifying it with selective PCR with oligonucleotides carrying phosphorothioated groups at 5′. A treatment with an exonuclease thus enables exclusive degradation of the template. In most preferred embodiments, transience is conferred by using a uracil-containing template such as mRNA. mRNA has a higher affinity of binding and can be removed by mRNA-specific enzymes. Such an mRNA template can be prepared in vivo or in vitro. In more preferred embodiments, use of an mRNA template entails including in the process at least three primers linked with a ligase.

In yet another preferred embodiment, the template enables orientation of multimolecular ligation of flush ends. In this embodiment, the template comprises a relatively short single- or double-stranded polynucleotide that is exactly complementary to the 3′ end of a first fragment and to the 5′ end of a second fragment that is adjacent to the first fragment in the parental polynucleotide. This facilitates adjacent hybridization of these two ends on the template.

Further embodiments include any or all of the following: the template and donor fragments are from different sources, the template is separately added to the reaction mixture, and/or the template is modified in specific ways to increase chimeragenesis.

Donor Fragments

Fragments can be recruited from homologous polynucleotides, related genes or from other genes. The parental DNA need not be characterized at all, but can be extracted from cells, clinical samples or the environment. Step (a) encompasses both starting with pre-fragmented single- or double-stranded fragments from an initial fragment-containing library, and/or starting with the substep of fragmenting single- or double-stranded parental polynucleotides from an initial library. Step (a) may comprise combining distinct libraries of fragments and/or fragmenting parental polynucleotides from distinct starting libraries. It may also comprise fragmenting parental polynucleotides from the same library in different ways, such as with different restriction enzymes. Furthermore, step (a) may comprise employing more fragments from one parental polynucleotide than another. For example, an experimenter using the process may bias the results by using more fragments of or parts of polynucleotide X than fragments of or parts of polynucleotide Y.

In one embodiment, supplemental single- or double-stranded fragments of variable length are added at steps (b) or (c). These supplemental fragments may substitute for some of the fragments of step (a), particularly if their sequences are homologous to the sequences of the step (a) fragments. Such supplemental fragments may, for example, introduce one or more direct mutations. They may also comprise synthetic sequences.

Fragmenting may occur before or after denaturing of the sequences that are fragmented. Fragmentation can be controlled or random. If random, any enzymatic or mechanical means known to those skilled in the art can be used to randomly cut the DNA, for example, digestion by DNase I or ultrasonication. If the fragmentation is controlled, it facilitates management over the degree, rate, efficiency and/or location of recombination. A preferred embodiment comprises hydrolyzing the parental polynucleotides with restriction enzymes to create restriction donor fragments. Restriction enzymes provide control over the degree, rate and efficiency of recombination by controlling the number of fragments produced per sequence. For example, the number may be increased by using restriction enzymes with many cutting sites or by using several different restriction enzymes. The greater the number of fragments produced per sequence, the greater the number (n) of fragments that must be recomposed to form a recombinant sequence. Preferably, n is 3 or more.

By controlling the nature and position of the fragment ends, restriction enzymes further provide control over not only degree and rate but also the location where recombination occurs. For example, the fragmenting can be designed so that the cuts occur in zones of the parent sequences that are homologous to zones in a reference sequence or an assembly template.

Fragments are preferably about 15-500 residues in length. When fragmentation is performed nonrandomly, the fragments are advantageously at least 15 residues in length and more preferably about 15-40 residues in length. The phrase “at least 15 residues” means between about 15 residues and the length of the longest polynucleotide used less one residue. When fragmentation is performed randomly, they are more preferably at least 50 residues in length. The phrase “at least 50 residues” means between about 50 residues and the length of the longest polynucleotide used less one residue.

The ends of at least two of the fragments at step (a) must be capable of being adjacently hybridized and ligated. (In ligation-only embodiments, all of the fragments that hybridize and form the final recombinant strand must have such ends.) In a preferred embodiment the invention employs flap trimming enzymes to make ligatable ends that would otherwise result in unproductive fragments. These enzymes recognize and degrade or cut in a specific way the nonhybridized ends of fragments when they cover other hybridized fragments on the same template.

A preferred enzyme is Flap endonuclease, which can be used at step (c) or during the hybridization of step (b). When the fragments are initially double-stranded, an embodiment of the invention comprises using specific exonucleases that recognize and degrade single-stranded sequences like the nonhybridized ends of the fragments. Such single-strand exonucleases or Flap endonucleases are preferably at a concentration (e.g., about 1.8-2.2 μg/ml of Flap endonuclease) that avoids their more general exonuclease activity, which could, for example, degrade the templates or recombinant sequences. These enzymes increase the number of fragment ends that can be ligated in step (c), which is particularly useful for randomly cut fragments because they tend to result in many overhanging flaps. Use of such enzymes with low hybridization temperatures and/or high hybridization times (e.g., two minutes) also facilitates recombination between low-homology polynucleotides. For example, a preferred embodiment that employs random fragmenting includes use of a Flap endonuclease and a wide range of hybridization temperature (e.g., from 5 to 65° C.) at step (b) that can be disconnected from step (c) ligation with regard to temperature, particularly when the hybridization temperature is lower than the high ligation temperature (e.g., about 60-75° C.). Most preferably, the Flap endonuclease concentration is about 2 μg/ml, the hybridization temperature is about 10° C. and the ligation temperature is about 65° C. When such trimming enzymes are employed, they are preferably thermoresistant, thermostable and active at high temperatures, like the ligase.

Additional Optional Features of the Method of the Invention

Unlike conventional shuffling methods, various embodiments of the invention do not require thermocycling, e.g., the repeated heating and cooling necessary for sexual PCR. In various embodiments, the process may be used to create gene-length polynucleotides or short polynucleotides. In various embodiments, hybridization may occur under conditions of low stringency. In various embodiments, the ratio between templates and chimeric polynucleotides produced is about 1. In various embodiments, no DNases are employed. In various embodiments, the initial library comprises variants of a single gene. In various embodiments, the initial library may comprise polynucleotides having artificially induced point mutations. In various embodiments, the invention may be used for whole genome shuffling. In various embodiments, the steps may occur in vivo rather than in vitro. Finally, when amplifying fragments by PCR, for example, the initiated sequences can be designed to produce fragments whose ends are adjacent all along the assembly template.

### EXAMPLE I

The object of Example I is to produce recombinant polynucleotides from the kanamycin resistance gene, using solitary-strand fragments.

First, the resistance gene (1 Kb) of pACYC184 is cloned in the polylinker of M13mp18 so that the solitary-strand phagemide contains the noncoding strand of the gene.

In parallel, this gene is amplified by PCR mutagenesis (error-prone PCR) with two initiators that are complementary to vector sequence M13mp18 on each side of the gene sequence. The initiator for the noncoding strand is phosphorylated while the initiator for the coding strand is not. The product of the PCR mutagenesis is digested by the lambda exonuclease, which produces a library of coding strands for mutants of the kanamycin resistance gene.

This library of solitary-strand sequences is digested by a mixture of restriction enzymes, notably Hae III, Hinf I and Taq I. The resulting solitary-strand fragments are then hybridized with the solitary-stranded phagemide and ligated with a thermostable ligase. This step is repeated several times until the small fragments can no longer be observed during deposition on an agarose gel. Meanwhile, the band corresponding to the solitary-stranded of the complete resistance gene becomes a major component of the “smear” visible on the gel.

The band corresponding to the size of the gene is cut from the gel and purified. It is then hybridized with two complementary oligonucleotides (40 mer) of the M13mp18 sequences on each side of the gene and this partial duplex is digested by Eco RI and Sph I, then ligated in an M13mp18 vector digested by the same enzymes.

The cells transformed with the ligation product are screened for increased resistance to kanamycin.

The cloning of solitary-stranded recombinant molecules can optionally be performed by PCR with two initiators of the complete gene and cloning of the double-stranded product of this amplification. To avoid undesirable mutations, this amplification should be performed with polymerase of the Pfu type and with a limited number of cycles.

The plasmids of the clones that are significantly more resistant to kanamycin than the initial stock are purified and used for PCR with the polymerase Pfu, under high fidelity conditions, with the phosphorylated/nonphosphorylated initiator couple as previously defined. This produces the second generation of solitary-stranded fragments after a treatment with lambda exonuclease and fragmentation with restriction enzymes. The enzymes used for this step can comprise a different mixture (e.g., Bst NI, Taq I and Mnl I).

The recombination and selection steps are repeated several times until a substantial increase in resistance to kanamycin is obtained.

### EXAMPLE II

I. Summary

The starting library included 10 gene mutants of ponB, coding for the PBP1b of E. coli (1). The sequence of each mutant differed from that of the native gene by a non-homologous zone 3-16 bases in length resulting from the substitution of five initial codons by five alanine codons, according to the technique described by Lefevre et al and incorporated herein (8).

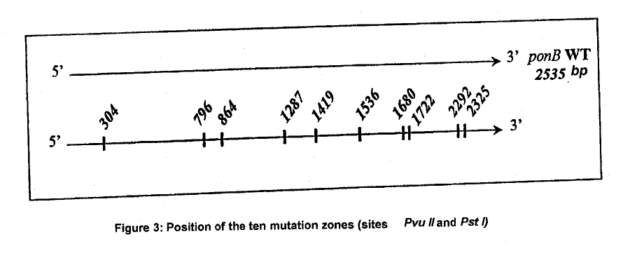

The substitution represented a unique site of the restriction enzyme Pvu II surrounded by two Pst I enzyme sites, which permitted the mutants to be distinguished from each other by their digestion profile. FIG. 3 represents the positions of the ten zones of mutations (Pvu II and Pst I) carried by each mutant.

After PCR amplification of the mutants, the PCR products were purified and mixed in equimolar quantity in order to form the library. The polynucleotide sequences of this library were digested with the restriction enzymes Hinf I and Bsa I, in such a way as to generate libraries of restriction fragments. The restriction fragments were then incubated with various amounts of the wild-type template, at different quantities, in the presence of a thermostable ligase. After several denaturation/hybridization/ligation cycles, a fraction of the reaction mixture was used to carry out a PCR amplification with a couple of primers specific to the 5′ and 3′ ends of the mutant genes and non-specific to the 5′ and 3′ ends of the wild-type template. The amplification product was cloned and the clones were analyzed for their digestion profile with the Pvu II or Pst I restriction endonucleases. The obtained profiles indicated which fragments of the mutants were able to be recombined with the others to form an entire gene.

II. Materials and Methods

A. Strains and Plasmids

The strain MC1061 (F⁻ araD139, Δ(ara-leu)7696, galE15, galK16, Δ(lac)X74, rpsL (Str^{R}), mcrA mcrB1, hsdR2 (rk⁻mk⁺)) is derived from Escherichia coli K12.

The vector pARAPONB stems from the vector pARA13 (3) in which the ponB gene carrying a thrombin-cutting site (9) was introduced between the restriction sites Nco I and Nar I. The vector pET26b+ is one of the pET vectors developed by Studier and Moffatt (10) and commercialized by NOVAGEN Corporation.

B. Oligonucleotides

The oligonucleotides were synthesized by ISOPRIM corporation (Toulouse). The oligonucleotide sequences are reported in Table I below.

TABLE I

| | |
|---|---|
| Oligo N | 5′ ACTGACTACCATGGCCGGGAATGACCGCGAGCC 3′ |
| Oligo E | 5′ CCGCGGTGGAGCGAATTCTAATTACTACCAAACATATCC |

3′

| | |
|---|---|
| Oligo M1 | 5′ GCGCCTGAATATTGCGGAGAAAAAGC 3′ |
| Oligo M2 | 5′ ACAACCAGATGAAAAGAAAGGGTTAATATC 3′ |
| Oligo A1 | 5′ ACTGACTACCATGGCC 3′ |
| Oligo A2 | 5′ CCGCGGTGGAGCGAATTC 3′ |

C. Reagents

The restriction and modification enzymes cited in Table II below were used according to the recommendations of the suppliers.

TABLE II

| Enzyme | Concentration | | Supplier |
|---|---|---|---|
| NcoI | 10 | U/μl | New England Biolabs |
| PstI | 20 | U/μl | New England Biolabs |
| Eco RI | 20 | μ/μl | New England Biolabs |
| Bsa I | 5 | U/μl | New England Biolabs |
| Hinf I | 10 | U/μl | New England Biolabs |
| Pvu II | 10 | U/μl | New England Biolabs |
| T4 DNA ligase | 400 | U/μl | New England Biolabs |
| Taq DNA polymerase | 5 | U/μl | PROMEGA |
| AMPLIGASE | 100 | U/μl | EPICENTRE |

The buffers used are reported in Table III below.

TABLE III

| Buffers | Composition |
|---|---|
| T | Tris HCl 10 mM, pH 8.0 |
| Polymerization 20X | Tris HCL 100 mM pH 8.3, MgCl₂ 15 mM, KCl 500 |
| | mM, 1.0% TRITON X100 ® |
| Restriction A 10X | 500 mM NaCl, 100 mM Tris HCl pH 7.9, 100 mM |
| | MgCl₂, 10 Mm DTT, |
| Restriction B 10X | 1 M NaCl, 500 mM Tris HCl pH 7.9, 100 mM |
| | MgCl₂, 10 mM DTT |
| Restriction C 10X | 500 mM NaCl, 1 M Tris HCl pH 7.5, 100 mM mM |
| | MgCl₂, 0.25% TRITON X100 ® |
| AMPLIGASE 10X | 200 mM Tris HCl pH 8.3, 250 mM KCl, 100 mM |
| | MgCl₂, 5 mM NAD, 0.1% TRITON X100 ® |
| Ligation 10X | 500 mM Tris HCl pH 7.5, 100 mM MgCl₂, 100 |
| | mM DTT, 10 mM ATP, 250 μg/ml BSA |

III. Preparation of Template

The wild type ponB gene was amplified by a PCR reaction step by using as primers the oligonucleotides M1 and M2 (FIG. 4). Five PCR reactions were prepared by adding 50 ng of pPONBPBR plasmid carrying the wild type gene (7) to a mixture containing 10 μl of polymerization buffer, 10 μl of dNTPs 2 mM, 20 pmol of each oligonucleotide M1 and M2, and 5U of Taq DNA polymerase, in a final volume of 100 μl. These mixtures were incubated in Perkin-Elmer 9600 Thermocycler according to the following program: (94° C.—2 min.)-(94° C. 15 sec.—60° C. 30 sec.—72° C. 1 min.)×29 cycles-(72° C.—3 min.).

The product of the five PCR was mixed and loaded on a 1% TBEagarose gelAfter migration and staining of the gel with ethidium bromide, the band at 2651 bp, corresponding to the ponB gene amplification product surrounded by two fragments of 26 bp and 90 bp respectively, was visualized by trans-illumination under ultraviolet, and cut out with a scalpel in order to be purified with the QUIAquick system (QIAGEN). All the DNA thus purified was eluted in 120 μl of buffer T. The concentration of this DNA was approximatively 100 ng/μl as measured by its absorbance at 260 nm.

IV. Preparation of the Library

A. Amplification of the Mutant Genes

The genes of the ten mutants were separately amplified by a PCR reaction with oligonucleotides N and E. These oligonucleotides introduce respectively the restriction sites Nco I and Eco RI, permitting the cloning of the products obtained with these two sites.

Each PCR reaction was prepared by adding 50 ng of the plasmid carrying the mutant gene to a mixture containing 10 μl of polymerization buffer, 10 μl of dNTPs 2 mM, 20 pmol of each oligonucleotide N and E, and 5U of Taq DNA polymerase, in a final volume of 100 μl. This mixture was incubated in a Perkin-Elmer 9600 thermocycler according to the following program: (94° C.—2 min.)-(94° C. 15 sec.—60° C. 30 sec.—72° C. 1 min.)×29 cycles-(72° C.—3 min.).

The specificity of the genetic amplification was verified by restriction profile with the Pvu II endonuclease, by incubating 5 μl of each PCR product 1 hour at 37° C. in a mixture containing 3 μl of restriction buffer A and 5U of the Pvu II enzyme in a final volume of 30 μL. 15 μl of that digestion reaction were loaded on a TBE 1% agarose gel. After migration and staining with ethidium bromide, the gel was exposed to ultraviolet. The visualization of the restriction fragments permitted confirmation of the specificity of the genetic amplification of each mutant gene.

In parallel, 3 μl of each PCR reaction were loaded on a TBE 1% agarose gel. After migration, the gel was treated as above. The intensity of each band permitted the assessment that the genetic amplifications had the same yield.

B. Creation of Libraries of Restriction Fragments.

50 μl of each of the ten PCR were mixed and loaded on a 1% TBE agarose gel. After migration and staining with ethidium bromide, the band at 2572 bp, corresponding to the amplification product of the genes of the ten mutants, was cut out with a scalpel and purified with the Quiaquick system (QIAGEN). All the DNA thus purified was eluted in 120 μl of buffer T. The concentration of this DNA was approximately 100 ng/μl according to its absorbance at 260 nm.

In order to generate the libraries of restriction fragments, 100 μl of this DNA were incubated for one hour at 50° C. in a mixture containing 12 μl of restriction buffer B, 1.2 μl of BSA (at 10 mg/ml), 25 U of the enzyme Bsa I and 4 μl of water. Then, 2 μl of restriction buffer B, 2 μl of BSA (at 1 mg/ml), 50 U of the enzyme Hinf I and 11.5 μl of water were added to the mixture, which was incubated for one hour at 37° C. The digestion mixture was purified on a QIAquick column (QIAGEN), and eluted with 30 μl of buffer T. 1 μl of this eluate was loaded on a 1% TBE agarose gel in order to verify that the digestion had been total, and that it had generated 6 restriction fragments, and consequently six libraries of fragments, of 590 bp, 500 bp, 472 bp, 438 bp, 298 bp and 274 bp. The concentration of this DNA was approximately 250 ng/μl according to its absorbance at 260 nm.

V. Recombining Ligation Reaction (RLR)

The RLR reaction was carried out by incubating determined quantities of restriction fragments Hinf I-Bsa I from the genes of ten mutants with the complete template (i.e., the wild type ponB gene), in the presence of a thermostable DNA ligase. The table IV below reports the composition of the mixtures for RLR.

TABLE IV

| | RLR 1 | RLR 2 | RLR 3 | RLR 4 | T- |
|---|---|---|---|---|---|
| Fragments Hinf I - Bsa I | 0.5 μl | 1 μl | 2 μl | 5 μl | 5 μl |
| of ten mutants | | | | | |
| (100 ng/μl) | | | | | |
| Wild type ponB template | 0.6 μl | 1.2 μl | 2.4 μl | 6 μl | 6 μl |
| (100 ng/μl) | | | | | |
| AMPLIGASE 10X | 2 μl | 2 μl | 2 μl | 2 μl | 2 μl |
| Buffer | | | | | |
| AMPLIGASE (25 U/μl) | 1 μl | 1 μl | 1 μl | 1 μl | — |
| H₂O | qsp | qsp | qsp | qsp | qsp |
| | 20 μl | 20 μl | 20 μl | 20 μl | 20 μl |

The negative control is identical to the reaction of RLR4, but does not contain thermostable DNA ligase. These different mixtures were covered with a drop of mineral oil and incubated in a Perkin-Elmer 9600 thermocycler in 200 μl microtubes according to the following program: (94° C., 5 min.)-(94° C., 1 min.—65° C., 4 min.)×35 cycles.

10 μl of each RLR reaction were then added to a PCR reaction mixture containing 10 μl of polymerization buffer, 10 μl of 2 mM dNTPs, 40 pmol of each oligonucleotide A1 and A2, and 5 U of Taq DNA polymerase in a final volume of 100 μl. This mixture was incubated in a Perkin-Elmer 9600 thermocycler according to the following program: (94° C., 5 min.)-(94° C., 30 sec.—46° C., 30 sec.—72° C., 1 min.)×29 cycles-(72° C., 2 min.). This PCR reaction permitted specific amplification of the ligation products formed in the course of the RLR reaction, without amplifying the template, since the oligonucleotides A1 and A2 are not able to hybridize with the template (it), as shown in FIG. 4.

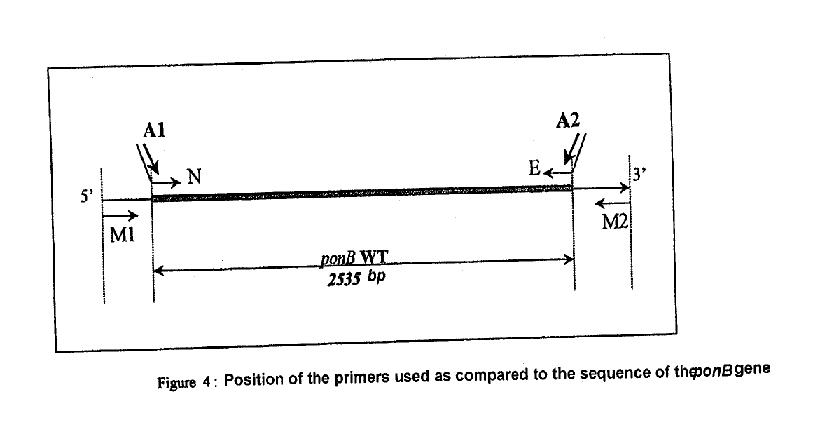

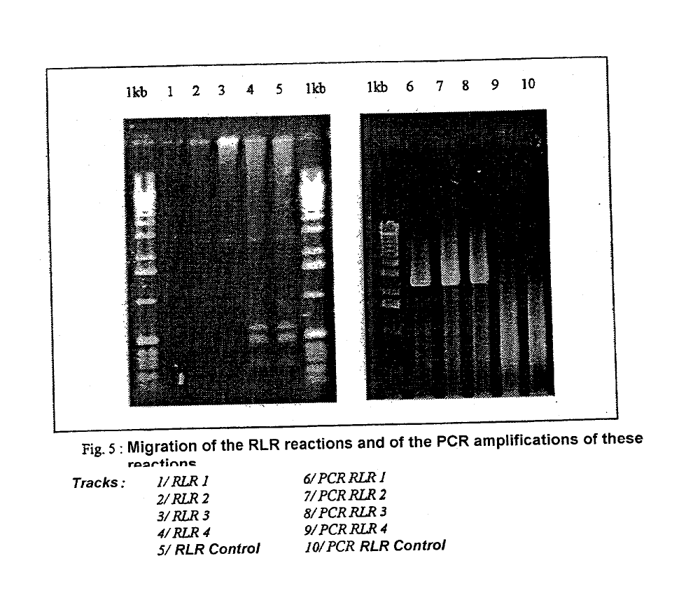

5 μl of each RLR reaction and 10 μl of each of the previous PCR reactions were loaded on a 1% TBE agarose gel. After staining with ethidium bromide, the gel was exposed to ultraviolet light, as shown in FIG. 5.

The analysis of this gel reveals that only the reaction of RLR4 contains, as the negative control, restriction fragments still visible (tracks 4 and 5).

The absence of PCR product for the negative control (track 10) reveals not only that the PCR reaction is specific (no amplification of the complete template), but also that the restriction fragments present in the mixture cannot be substituted for the primers to generate a contaminant PCR product under the chosen conditions. In parallel, the presence of a unique band at about 2500 bp in tracks 6, 7 and 8 demonstrates that an RLR product was able to be amplified by PCR for the RLR1, 2 and 3 reactions. These three RLR reactions therefore permitted the regeneration of one or more of the complete genes starting from six libraries of restriction fragments.

VI. Analysis of the Amplification Products

A. Cloning

The PCR amplification products of the RLR 1, 2 and 3 reactions were purified with the Wizard PCR Preps system (PROMEGA) and eluted in 45 μl of buffer T. 6 μl of each purified PCR were incubated 1 hour at 37° C. in a mixture containing 3 μl of restriction buffer C, 3 μl of BSA (1 mg/ml), 20 U of the Eco RI enzyme, 10 U of the Nco I enzyme and 15 μl of water.

In parallel, two vectors (PARAPONB and pET26b+) were prepared for the cloning. These vectors were linearized by incubating 3 μg of these plasmids for 2 hours at 37° C., in a mixture containing 3 μl of restriction buffer C, 3 μl of BSA (1 mg/ml), 20 U of the Eco RI enzyme, 10 U of the Nco I enzyme and 19 μl of water.

The linearized vectors as well as the digested PCR were purified on a TBE 1% agarose gel with the QIAquick system (QUIAGEN). Each vector or each digested PCR was eluted in 30 μl of buffer T.

The ligation of each PCR digested with each of the vectors was carried out according to the conditions described in table V below, and incubated at 16° C. for 16 hours.

TABLE V

| | Ligation with the vector | | | | | | | |
|---|---|---|---|---|---|---|---|---|
| | pARAPONB | | | | Ligation with the vector pET26b+ | | | |
| | LpAR 1 | LpAR 2 | LpAR 3 | TlpAR | LpET1 | LpET2 | LpET3 | TLpET |
| PCR | 4 μl | — | — | — | 4 μl | — | — | — |
| amplification | | | | | | | | |
| RLR 1 digested | | | | | | | | |
| Nco I - Eco RI | | | | | | | | |
| PCR | — | 4 μl | — | — | — | 4 μl | — | — |
| amplification | | | | | | | | |
| RLR 2 digested | | | | | | | | |
| Nco I - Eco RI | | | | | | | | |
| PCR | — | — | 4 μl | — | — | — | 4 μl | — |
| amplification | | | | | | | | |
| RLR 3 digested | | | | | | | | |
| Nco I - EcoRI | | | | | | | | |
| Vector | 1 μl | 1 μl | 1 μl | 1 μl | — | — | — | — |
| pARAPONB | | | | | | | | |
| digested | | | | | | | | |
| Nco I - Eco RI | | | | | | | | |
| Vector pET26b+ | — | — | — | — | 1 μl | 1 μl | 1 μl | 1 μl |
| digested | | | | | | | | |
| Nco I - Eco RI | | | | | | | | |
| Ligation Buffer | 2 μl | 2 μl | 2 μl | 2 μl | 2 μl | 2 μl | 2 μl | 2 μl |
| Ligase | 1 μl | 1 μl | 1 μl | 1 μl | 1 μl | 1 μl | 1 μl | 1 μl |
| H₂O | 12 μl | 12 μl | 12 μl | 16 μl | 12 μl | 12 μl | 12 μl | 16 μl |

200 μl of chimiocompetent MC1061 cells (4) were transformed with 10 μl of each ligation by a thermal shock (5), and the cells thus transformed were spread over a selection medium.

No clone was obtained after transformation of ligation controls TLpAR and TLpET, thus indicating that the Nco I-Eco RI vectors pARAPONB and pET26b+ cannot undergo an intramolecular ligation.

B. Screening by PCR

A first screening of the clones obtained after transformation of the ligations with the vector pARAPONB was carried out by PCR. 42 colonies, 14 from each ligation LpAR1, LpAR2 and LpAR3, were resuspended individually in a PCR mixture containing 5 μl of polymerization buffer, 40 pmol of each oligonucleotide A1 and A2, 5 μl of 2 mM dNTPs and 5U of Taq DNA polymerase in a final volume of 50 μl. A negative control was obtained by adding to the PCR mixture 50 ng of the plasmid pBR322 in place of the colony. These 43 tubes were incubated in a Perkin-Elmer 9600 thermocycler according to the following program: (94° C., 5 min.)-(94° C., 30 sec.—46° C., 30 sec.—72° C., 1 min.)×29 cycles-(72° C., 2 min.). 5 μl of each of these PCR reactions were then incubated for 1 hour at 37° C. in a mixture containing 2 μl of restriction buffer A, 2 μl of BSA (1 mg/ml) and 5 U of the restriction enzyme Pvu II in a final volume of 20 μl.

10 μl of each of these digestions were loaded on a TBE 1% agarose gel in parallel with 5 μl of each non-digested PCR (thus avoiding possible confusion of non-specific bands of the PCR with a fragment obtained by restriction digestion). After migration and staining of this gel with ethidium bromide, the bands resulting from the digestion by the enzyme Pvu II were analyzed in order to determine which fragment(s) of initial mutants was/were associated with the others in order to reconstruct an entire gene. This screening reveals the presence of 27 genes carrying one mutation, 7 genes carrying two mutations and 8 genes no longer carrying any mutation.

C. Screening by Plasmidic DNA Minipreparation

The second screening was carried out by extracting the plasmidic DNA (5) from 21 clones resulting from the transformation of the ligations with the vector pET26b+ (7 clones of each ligation). 5 μl of the plasmidic DNA thus obtained for each clone were incubated for 1 hour at 37° C. in a mixture containing 1 μl of restriction buffer C, 6 U of the enzyme Pst I, 3 U of the enzyme Nco I and 6 U of the enzyme Eco RI in a final volume of 10 μl. 5 μl of each of these digestions were loaded on a TBE 1% agarose gel. After migration and staining of this gel with ethidium bromide, the bands resulting from the digestion by the Pst I enzyme were analyzed in order to determine which fragment(s) of the initial mutants had associated with the others in order to reconstruct an entire gene. This screening reveals the presence of 13 genes carrying a mutation, 5 genes carrying two mutations and 3 genes no longer carrying a mutation.

D. Statistical Analysis of the Recombinations.

In view of the position of each mutation with regard to the cutting sites of the enzymes Hinf I and Bsa I (see FIG. 6), it is possible to calculate the probability of obtaining through RLR a gene carrying 0, 1, 2, 3, or 4 of the mutations of the initial genes.

Assuming that the RLR reaction is totally random, the probabilities P are as follows:

$$\begin{matrix}P ( 0 mutation ) = {\prod }_{i = 6}^{9} ( \frac{i}{10} ) = 30.24 \% \\ P ( 1 mutation ) = {\sum }_{n = 1}^{4} [ \frac{n}{10 - n} {\prod }_{i = 1}^{4} ( \frac{10 - i}{10} ) ] = 44.04 \% \\ \begin{matrix}P ( 2 mutations ) = {\sum }_{n = 1}^{4} [ {\sum }_{a = 1}^{4 - n} ( \frac{10 - a}{a} ) ( \frac{10 - ( a + n )}{a + n} ) {\prod }_{i = 1}^{4} ( \frac{i}{10} ) ] = 21.44 \%\end{matrix} \\ P ( 3 mutations ) = {\sum }_{n = 1}^{4} [ ( \frac{10 - n}{n} ) {\prod }_{i = 1}^{4} ( \frac{i}{10} ) ] = 4.04 \% \\ P ( 4 mutations ) = {\prod }_{i = 1}^{4} ( \frac{i}{10} ) = 0.24 \%\end{matrix}$$

The two screenings carried out give results close to these statistical predictions, as reported in table VI below, thus indicating that the RLR reaction is quasi-random. A slightly higher proportion of genes carrying one mutation, to the detriment of the genes carrying zero mutation, is observed. This phenomenon could be attributed to a weak toxicity of the ponB gene already observed and to the slight of expression leakage of vectors pARAPONB and pET26b+, which would favor the selection of genes carrying an inactivating mutation.

TABLE IV

| | | | 2 | 3 | 4 |
|---|---|---|---|---|---|
| % | 0 mutation | 1 mutation | mutations | mutations | mutations |
| Statistics | 30.24 | 44.04 | 21.44 | 4.04 | 0.24 |
| PCR | 21 | 63 | 16 | 0 | 0 |
| Screening | | | | | |
| Mini- | 14 | 62 | 24 | 0 | 0 |
| preparation | | | | | |
| Screening | | | | | |

### EXAMPLE III

Example III depicts an embodiment of the invention that employs controlled digestion.

I. Materials and Methods

A. Bacterial Strains, Genomic and Plasmid DNA

For all DNA manipulations, standard techniques and procedures were used. E coli MC1061DE3 cells were used to propagate the expression plasmid pET26b+ (Novagen).

B. Oligonucleotides

All synthetic oligonucleotide primers for PCR were synthetized by MWG Biotech. The sense primer 5′ AGGAATTCCATATGCGAAAGAAAAGACGGGGA 3′ and the antisense primer 5′ ATAAAGCTTTCACTTGATGAGCCTGAGATTTC 3′ were used to amplify the Thermotoga Neapolitana Xylanase A gene and introduce NdeI and HindIII restriction sites (underlined). The NdeI site contained the initial codon (boldface).

C. Enzymes

Restriction enzymes, DNA polymerases and thermostable ligase were purchased from NEB and EPICENTRE and used as recommended by the manufacturers.

D. DNA Amplification, Cloning and Expression

PCR amplifications were carried out on a PE 9600 thermocycler. The Thermotoga Neapolitana Xylanase A amplicon was digested with primer-specific restriction endonucleases, ligated into compatible site on pET26b+, and transformed into E coli MC1061DE3. The MC1061DE3 clone containing the pET26b+XynA expression vector was propagated at 37° C. in LB containing kanamycin (60 μg/ml).

E. Biochemical Characterization

Thermal inactivation experiments were performed directly on E coli expressing XynA. Cells were re-suspended, after centrifugation at 6000 g for 5 min at 4° C., in 200 mM acetate buffer pH 5.6. Re-suspension was performed with an appropriate volume in order to standardize the amount of cell per sample. 150 μl of cells were then incubated at the appropriate temperature during different times. 100 μl of these cells were added to 100 μl of 0.5% (w/v) of xylan in 200 mM acetate buffer pH 5.6 and incubated 10 min at 80° C. Then, 200 μl of 3,5-Dinitrosalicylic acid were added and boiled 5 min, refrigerated 5 min on ice and centrifuged 5 min at 12000 g. 150 μl were transferred in μtiterplate and OD at 540 nm was measured.

For optimal temperature experiments, 100 μl of 0.5% (w/v) of xylan in 200 mM acetate buffer pH 5.6, were added to 100 μl of resuspended cells and incubated for 10 min at different temperatures during the 10 min. Then, 200 μl of 3,5-dinitrosalicylic acid were added and boiled for 5 min, refrigerated for 5 min on ice and centrifuged for 5 min at 12000 g. 150 μl were transferred in μtiterplate and OD at 540 nm was measured.

II. Results

A. Generation of Low Thermostable Mutant of XynA

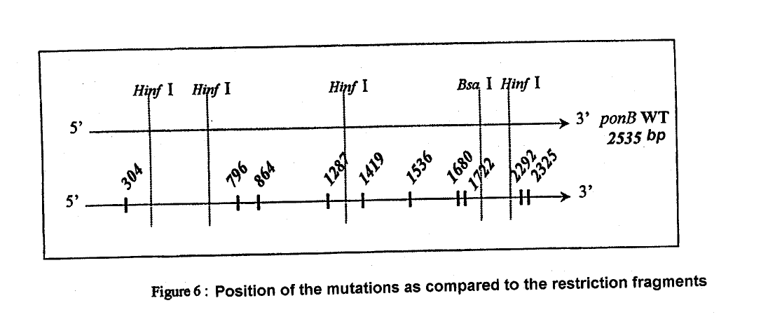

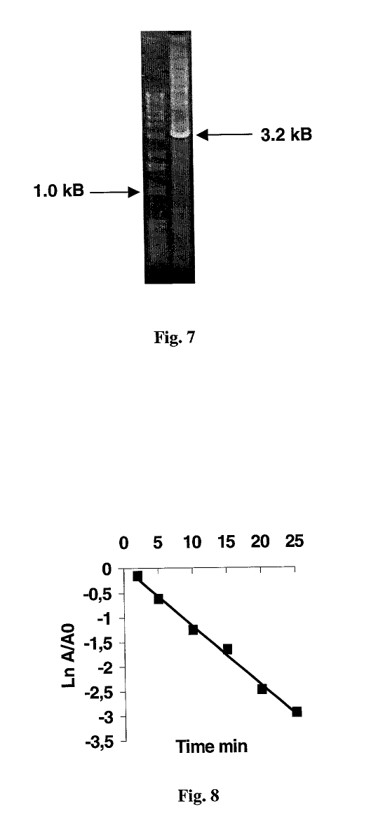

To generate a low thermostable mutant of XynA protein, error-prone PCR was performed as shown in FIG. 7, Error-prone PCR on WT XynA gene, using 1% agarose gel. The products were digested with primer-specific restriction endonucleases, ligated into compatible sites on pET26b+, and transformed into E coli MC1061DE3 to generate an error-prone library.

One clone (mutant 33) from the error-prone library seemed to have very low thermostability compared to the WT protein. A rapid biochemical analysis, including determination of an optimal temperature and thermal inactivation, was done and compared to the WT one. Regarding the optimal temperature, mutant 33 had an optimal temperature around 78° C. compared to the WT one (above 90° C.) but, for mutant 33 no residual activity was detected after 30 min incubation at 82° C. or 1 min at 95° C. and the inactivation constant calculated from FIG. 8, Thermal inactivation of mutant 33 at 82° C., was estimated at 0,120 min⁻¹ at 82° C. No or low thermal inactivation was detected for the WT protein at these temperatures.

B. Shuffling Experiments

The mutant 33 and WT genes were then recombined using L-Shuffling™ technology to generate mutants with different thermostabilities. Different mutants were expected: mutants with WT optimal temperature, mutants with lower thermostability than WT and mutants with higher thermostability than that of the mutant 33's optimal temperature.

1) Fragments Library

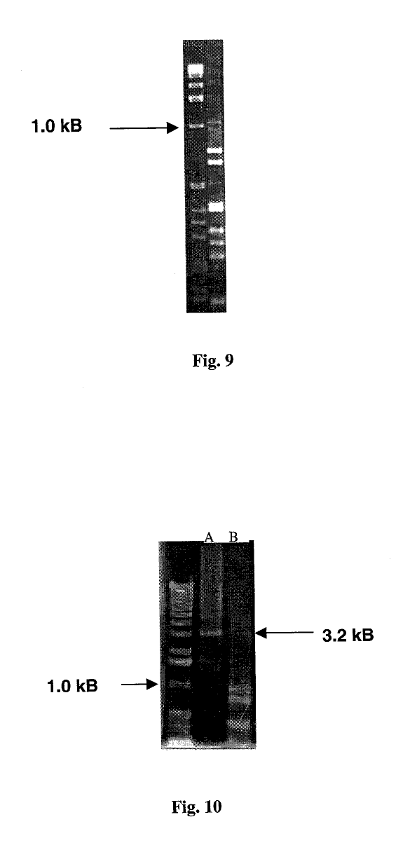

After PCR amplification of WT and mutant 33, the products were digested with a mix of six restriction enzymes, HincII, BamHI, XhoI, SphI, EcoRI, EcoRV, generating eight fragments (from 120 to 700 pb). See FIG. 9, Fragmentation of PCR products with a mix of six restriction endonucleases, using 3% agarose gel.

2) Shuffling Experiment

RLR (recombining ligation reaction) was performed with standardized fragments (shown in FIG. 9) and NdeI/HindIII digested pET26+XynA as template with the thermostable ligase using several cycles of denaturation and hybridation/ligation steps.

A negative control was done with the same conditions without the thermostable ligase (B) and the results are shown in FIG. 10, L-Shuffling™ experiments using 1% agarose gel. FIG. 10 shows that without thermostable ligase, the fragments are not used for any recombination. A selective digestion of the template was then performed by adding DpnI to the reaction mixture.

3) Cloning Products

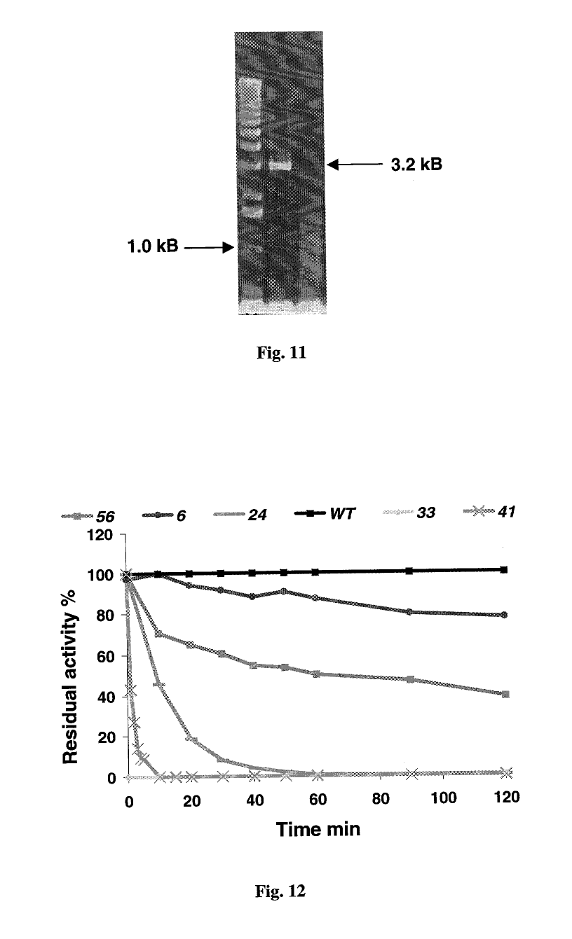

A PCR Pfu amplification (FIG. 11, PCR Pfu on L-Shuffling™ products using 1% agarose gel) was performed on DpnI digested L-shuffling™ products both for A and B (negative control, FIG. 9) using 5′ sense and 5′ antisense synthetic primers and the protocol described above. No template amplification occurred, despite obtaining a large amount of amplified L-shuffling™ products for cloning. For this, L-shuffling™ products were digested with primer-specific restriction endonucleases, ligated into compatible sites on pET26b+, and transformed into E coli MC1061DE3 to generate a L-Shuffling™ library.

4) Biochemical Characterization

Several clones were selected from the L-Shuffling™ library for activity remaining after 30 min incubation at 82° C.

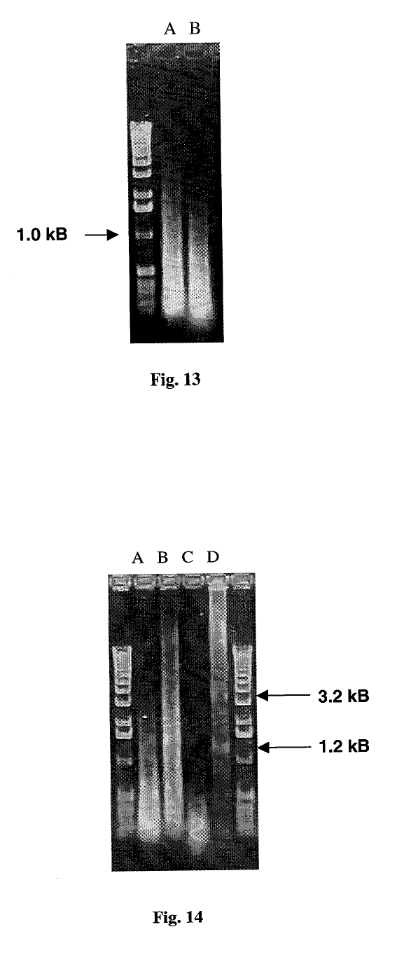

Clones 24, 41 and 56 (FIG. 12, Thermal inactivation of mutants at 95°) have the optimal temperature of mutant 33, and clone 6 has the optimal temperature of the WT xylanase. In these experimental conditions, WT xylanase retained 100% of activity after 120 min incubation at 95° C. On the contrary, for mutant 33 no residual activity was detected after 1 min at 95° C. FIG. 13 shows four mutants from the L-Shuffling™ library that exhibited characteristics that differ from those of the two parents.

### EXAMPLE IV

Example IV depicts an embodiment of the invention that employs random digestion.

I. Materials and Methods

A. Bacterial Strains, Genomic and Plasmid DNA

For all DNA manipulations, standard techniques and procedures were used. E coli MC1061DE3 cells were used to propagate the expression plasmid pET26b+ (Novagen).

B. Oligonucleotides

All synthetic oligonucleotide primers for PCR were synthetized by MWG Biotech. The sense primer 5′ AGGAATTCCATATGCGAAAGAAAAGACGGGGA 3′ and the antisense primer 5′ ATAAAGCTTTCACTTGATGAGCCTGAGATTTC 3′ were used to amplify the Thermotoga Neapolitana Xylanase A gene and introduce NdeI and HindIII restriction sites (underlined). The sense primer 5′ GGAATTCCATATGGCGGCGGCAGCCGGCA 3′ and the antisense primer 5′ GGAATTCCTACTGCCGCTCCGATTGTGG 3′ were used to amplify the Acidobacterium capsulatum Xylanase gene and introduce NdeI and EcoRI restriction sites (underlined). The NdeI site contained the initial codon (boldface).

C. Enzymes

Restriction enzymes, DNA polymerases and thermostable ligase were purchased from NEB and EPICENTRE, and used as recommended by the manufacturers.

II. Results

The Thermotoga neapolitana gene (3.2 kB) and Acidobacterium capsulatum gene (1.2 kB) were recombined.

A. Fragments Library

PCR amplification on Thermotoga neapolitana and Acidobacterium capsulatum genes were performed, followed by digestion with DNaseI. See FIG. 13, DNaseI fragmentation of Thermotoga neapolitana (A) and Acidobacterium capsulatum (B) genes, using 1% agarose gel.

B. Shuffling Experiment

RLR was performed with standardized fragments (shown in FIG. 13) with thermostable ligase and thermostable flap, via several cycles of denaturation and hybridation/ligation.

Negative controls were performed under the same conditions but without the thermostable ligase and/or thermostable flap (A, B and C). The results are shown in FIG. 14, L-Shuffling™ experiments, using 1% agarose gel. FIG. 14 shows that without thermostable ligase and thermostable flap, the fragments are not recombined. In FIG. 14, A represents fragments without ligase and Flap activities; B represents fragments with only ligase; C represents fragments with only flap; and D represents the shuffling conditions.

### EXAMPLE V

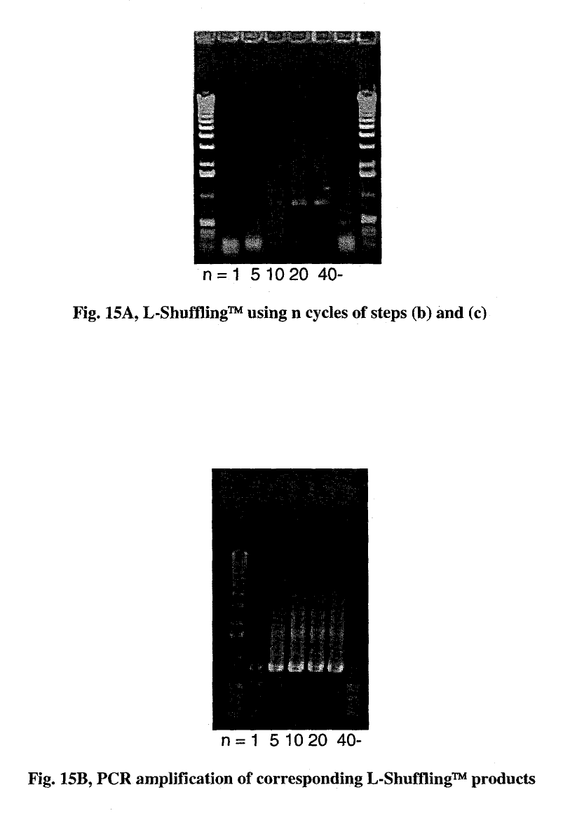

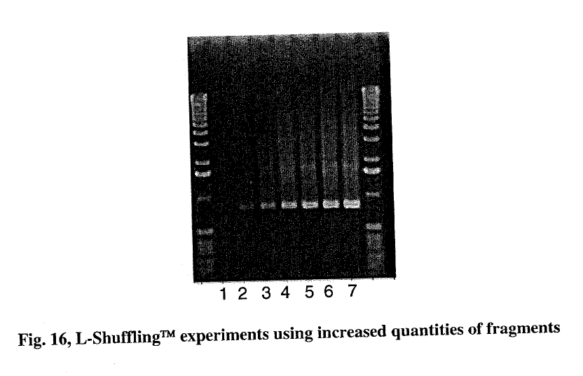

Example V employed the materials and methods of Example III but experimented with different numbers of cycles of steps (b) and (c). See FIG. 15A, L-Shuffling™ using n cycles of steps (b) and (c), and FIG. 15B, PCR amplification of corresponding L-Shuffling™ products. As shown in FIGS. 15A-B, at least one cycle (n=1) is necessary to obtain a recombinant polynucleotide.

### EXAMPLE VI

Example VI employed the materials and methods of Example III but experimented with seven quantities of fragments, as follows:

1:1×

2:2×

3:3×

4:4×

5:11×

6:14×

7:17×

FIG. 16, L-Shuffling™ experiments using increased quantities of fragments, shows the results for these seven quantities.

The foregoing presentations are not intended to limit the scope of the invention. Although illustrative embodiments of the present invention have been described in detail and with reference to accompanying drawings, it is obvious to those skilled in the art that modifications to the methods described herein can be implemented. These and other various changes and embodiments may be effected by one skilled in the art without departing from the spirit and scope of the invention, which is intended to be determined by reference to the claims and their equivalents in light of the prior art.

### BIOGRAPHICAL REFERENCES

1) Broome-Smith J. K., Edelman Al, Yousif S. and Spratt B. G., (1985), The nucleotide sequence of the ponA and ponB genes encoding penicillin-binding proteins 1A and 1B of Escherichia coli K12, Eur. J. Biochem., 147, 437-446.

2) Caldwell R. C. and Joyce G., 1992, Randomization of genes by PCR mutagenesis, PCR Methods and Application, 2, 28-33.

3) Cagnon C., Valverde V. and Masson J.-M., (1991), A new family of sugar inducible expression vectors for Escherichia coli, Prot. Eng., 4, 843-847.

4) Hanahan D., (1985), Techniques for transformation of Escherichia coli, in DNA cloning: a practical approach, Glover D. M. (ed), IRL Press, Oxford vol I, 109-135.

5) Maniatis T., Fristch E. F. and Sambrook J., (1982), Molecular cloning. A laboratory Manual, Cold Spring Harbor Laboratory, Cold Spring Harbor, N.Y.

6) Landt et al., Gene, 96, 125-128, 1990.

7) Lefevre F., Topological Analysis of the Penicillin Binding Protein 1b of Escherichia coli, 1997, These.

8) Lefevre F., Rémy M. H. and Masson J. M., 1997 (a), Alanine-stretch scanning mutagenesis: a simple and efficient method to probe protein structure and function, Nuc. Acids Res., 25, 447-448.

9) Lefevre F., Rémy M. H. and Masson J. M., 1997 (b), Topographical and functional investigation of Escherichia coli Penicillin-Binding Protein 1b by alanine stretch scanning mutagenesis, J. Bacteriol., 179, 4761-4767.

10) Studier F. W. and Moffatt B. A., 1986, Use of bacteriophage T7 RNA polymerase to direct selective high-level expression of cloned genes, J. Mol. Biol, 189, 113-130.

1. A template-mediated, ligation-oriented method for nonrandomly shuffling polynucleotides, comprising:
a) obtaining, directly or indirectly from a polynucleotide library, single-stranded fragments of at least two homologous polynucleotides;
b) hybridizing said fragments to one or more devised assembly templates until at least two of the fragments are adjacently hybridized, thereby forming at least one partially double-stranded polynucleotide, wherein at least one of said templates shares at least one zone of homology with said homologous polynucleotides;
c) treating said partially double-stranded polynucleotide to form at least one recombinant polynucleotide,
wherein said treating comprises, in any order, the following:
(i) ligating nicks, and
(ii) where necessary, any one of or any combination of the following gap filling techniques:
filling in gaps by further hybridizing said fragments to said templates to increase the number of fragments that are adjacently hybridized,
filling in short gaps by trimming any overhanging flaps of any partially hybridized fragments, and
filling in short gaps via polymerization.

2. The method of claim 1, wherein the steps occur in vitro.

3. The method of 1, wherein said homologous polynucleotides are double-stranded.

4. The method of claim 1, wherein steps (b) and (c) are carried out simultaneously.

5. The method of claim 1, wherein a recombinant polynucleotide is formed after one cycle of the method.

6. The method of claim 1, wherein a recombinant polynucleotide is formed after more than one repetition of step (a), (b) and/or (c).

7. The method of claim 6, wherein a recombinant polynucleotide is formed after more than three repetitions of step (a), (b) and/or (c).

8. The method of claim 1, wherein a recombinant polynucleotide is formed after more than three repetitions of steps (b) and (c).

9. The method of claim 1, wherein the templates or the homologous polynucleotides used in a subsequent cycle of the method are recombinant polynucleotides created by a prior cycle of the method.

10. The method of claim 1, wherein said templates are initially double-stranded.

11. The method of claim 1, further comprising treating the template strand of the recombinant polynucleotide to eliminate, separate or degrade said template strand.

12. The method of claim 11, wherein the recombinant polynucleotide is separated from the template strand due to a label on the template strand or on the recombinant strand.

13. The method of claim 11, wherein the templates comprise uracil.

14. The method of claim 13, wherein the templates are mRNA sequences.

15. The method of 1, wherein the fragments are initially double-stranded.

16. The method of claim 1, wherein supplemental or substitute fragments are added at step (a), (b) or (c).

17. The method of claim 1, wherein step (a) comprises fragmenting said homologous polynucleotides with at least one restriction enzyme which has multiple cutting sites on said homologous polynucleotides, or with a plurality of different restriction enzymes.

18. The method of claim 17, wherein the fragments at step (a) are at least 15 residues in length.

19. The method of claim 18, wherein the fragments at step (a) are about 15-40 residues in length.

20. The method of claim 1, wherein step (a) comprises obtaining at least two populations of fragments from distinct polynucleotide libraries, or obtaining at least two distinct populations of fragments from the same polynucleotide library using different restriction enzymes.

21. The method of claim 1, wherein step (a) comprises fragmenting said homologous polynucleotides randomly with DNase I, and wherein said homologous polynucleotides or the fragments obtained therefrom are initially double-stranded.

22. The method of claim 21, wherein the fragments at step (a) are at least 50 residues in length.

23. The method of claim 1, wherein the fragments of step (a) are amplified.

24. The method of claim 23, wherein the fragments of step (a) are amplified using oligonucleotide primers that generate fragments having ends adjacent along the whole length of the templates.

25. The method of claim 1, wherein step (a) comprises fragmenting the homologous polynucleotides into three or more fragments.

26. The method of claim 1, wherein a Flap endonuclease is added for use at step (b) and/or step (c).

27. The method of claim 26, wherein said Flap endonuclease has the same thermoresistance and high-temperature activity as a ligase used at step (c).

28. The method of claim 27, wherein the concentration of Flap endonuclease is about 1.8 to 2.2 μg/ml, the hybridizing occurs at a temperature of approximately 5-20° C., and the ligating occurs at a temperature of approximately 60-75° C.

29. The method of claim 1, wherein the polynucleotide library is generated from a native gene by successive directed mutagenesis, by error-prone PCR, by random chemical mutagenesis, by in vivo random mutagenesis, or by combining genes from gene families within the same or different species, thereby resulting in a variety of sequences in said polynucleotide library.

30. The method of claim 1, wherein the polynucleotide library comprises synthetic sequences or wherein synthetic fragments are added at step (a), (b) or (c).

31. The method of claim 1, wherein the templates are obtained from the polynucleotide library or from a consensus sequence of said library.

32. The method of claim 1, wherein polynucleotides, complementary to the 3′ end of one fragment and to the 5′ end of an adjacent fragment, are used as the templates.

33. The method of claim 1, wherein the recombinant polynucleotide is obtained without use of a polymerase

34. The method of claim 1, wherein the recombinant polynucleotide is obtained without inducing crossovers or strand switching.

35. The method of claim 1, wherein fragments and recombinant polynucleotides are obtained without size fractionation.

36. The method of claim 1, wherein step (b) or (c) entails multiple hybridization events.

37. The method of claim 1, wherein step (b) entails a single hybridization event.

38. The method of claim 1, wherein step (b) entails a single hybridization event and step (c) entails no hybridization event.

39. The method of claim 1, wherein the polynucleotides library or the templates are recombinant polynucleotides.

40. The method of claim 1, wherein said single-stranded fragments are solitary-stranded fragments.

41. The method of claim 1, wherein said templates are solitary-stranded templates.

42. The method of claim 1, wherein said fragments and said templates are solitary-stranded.

43. The method of claim 42, wherein the solitary-stranded fragments are from the top strand of said homologous polynucleotides and the solitary-stranded templates are from the bottom strand of said homologous polynucleotides.

44. The method of claim 42, wherein the solitary-stranded fragments are from the bottom strand of said homologous polynucleotides and the solitary-stranded template is from the top strand of said homologous polynucleotides.

45. The method of claim 1, wherein all or substantially all of the templates are longer than the fragments of step (a).

46. The method of claim 1, wherein the template strand of said recombinant polynucleotide is eliminated, separated or degraded.

47. The method of claim 1, wherein the homologous polynucleotides are 30-90% homologous to each other.

48. The method of claim 1, wherein all or substantially all of the templates are longer than the fragments of step (a).

49. The method of claim 1, wherein the templates are substantially equally homologous to each of said homologous polynucleotides.

50. The method of claim 1, wherein at least one-half of the homologous polynucleotides differ from each other in length by more than two residues.

51. The method of claim 1, wherein at least one-half of the homologous polynucleotides are only about 20-45% homologous to each other.

52. The method of claim 1, further comprising translating the recombinant polynucleotide in vitro to express any protein thereof.

53. The method of claim 1, further comprising step (d) selecting at least one of said recombinant polynucleotides that has a desired property.

54. The method of claim 53, wherein the steps occur in vitro.

55. The method of claim 53, wherein said single-stranded fragments are solitary-stranded fragments.

56. The method of claim 53, wherein the templates are solitary-stranded templates.

57. The method of claim 53, wherein any short gaps are filled by trimming overhanging flaps of partially hybridized fragments.

58. The method of claim 53, wherein all or substantially all of the templates are longer than the fragments of step (a).

59. The method of claim 53, further comprising translating the recombinant polynucleotide in vitro to express any protein thereof.

60. The method of claim 53, further comprising amplifying the recombinant polynucleotide before step (d).

61. The method of claim 53, wherein the template strand is eliminated, separated or degraded before step (d).

62. The method of claim 61, further comprising before step (d), but after eliminating, separating or degrading the template strand, re-creating a double-stranded recombinant polynucleotide from the recombinant strand and then cloning said double-stranded recombinant polynucleotide.

63. A template-mediated, ligation-oriented method for in vitro nonrandom shuffling of polynucleotides, comprising:
a) obtaining, directly or indirectly from a polynucleotide library, solitary-stranded restriction fragments of at least two homologous polynucleotides;
b) hybridizing said fragments to one or more devised assembly templates until at least two of the fragments are adjacently hybridized before any gap filling occurs, thereby forming at least one partially double-stranded polynucleotide, wherein all or substantially all of said templates are solitary-stranded, transient, longer than said fragments and share at least one zone of homology with said homologous polynucleotides;
c) treating said partially double-stranded polynucleotide to form at least one recombinant polynucleotide,
wherein said treating comprises, in any order, the following:
(i) ligating nicks, and
(ii) where necessary, any one of or a combination of the following gap filling techniques:
filling in gaps by further hybridizing said fragments to said templates to increase the number of fragments that are adjacently hybridized, and
filling in short gaps by trimming any overhanging flaps of any partially hybridized fragments; and
d) selecting at least one of said recombinant polynucleotides that has a desired property.

64. The method of claim 63, wherein the short gaps are filled by trimming overhanging flaps of partially hybridized fragments.

65. A template-mediated, ligation-only method for in vitro nonrandom shuffling of polynucleotides, comprising:
a) obtaining, directly or indirectly from a polynucleotide library, solitary-stranded restriction fragments of at least two homologous polynucleotides;
b) iteratively hybridizing said fragments to one or more devised assembly templates until all of the fragments that are hybridized to the templates are adjacently hybridized, wherein said templates are solitary-stranded and transient, and wherein at least one of said templates shares at least one zone of homology with said homologous polynucleotides;
c) ligating nicks to form at least one recombinant polynucleotide; and
d) selecting at least one of said recombinant polynucleotides that has a desired property.

66. A template-mediated, ligation-oriented method for in vitro nonrandom shuffling of mutation-containing zones of polynucleotides, comprising:
a) locating restriction sites for mutation-containing zones among polynucleotide alleles;
c) obtaining, directly or indirectly from said alleles, fragments corresponding to said restriction sites;
b) hybridizing said fragments to one or more devised assembly templates until at least two of the fragments are adjacently hybridized, thereby forming at least one partially double-stranded polynucleotide, wherein all or a portion of at least one of said templates is homologous to said mutation containing zones of said alleles;
c) treating said partially double-stranded polynucleotide to form at least one recombinant polynucleotide,
wherein said treating comprises, in any order, the following:
(i) ligating nicks, and
(ii) any one of or a combination of the following gap filling techniques:
filling in gaps by further hybridizing said fragments to said templates to increase the number of fragments that are adjacently hybridized, and
filling in short gaps by trimming any overhanging flaps of any partially hybridized fragments; and
d) selecting at least one of said recombinant polynucleotides that has a desired property.

67. A template-mediated, ligation-oriented method for in vitro nonrandom low-homology shuffling of gene families, comprising:
a) obtaining, directly or indirectly from a gene family library, solitary-stranded restriction fragments of at least two of said polynucleotides of said gene family;
b) hybridizing said fragments to one or more devised assembly templates until at least two of the fragments are adjacently hybridized, thereby forming at least one partially double-stranded polynucleotide, wherein said templates are solitary-stranded and transient, and wherein at least one of said templates shares at least one zone of homology with said gene family polynucleotides;
c) treating said partially double-stranded polynucleotide to form at least one recombinant polynucleotide,
wherein said treating comprises, in any order, the following:
(i) ligating nicks, and
(ii) any one of or a combination of the following gap filling techniques:
filling in gaps by further hybridizing said fragments to said templates to increase the number of fragments that are adjacently hybridized, and
filling in short gaps by trimming any overhanging flaps of any partially hybridized fragments; and
d) selecting at least one of said recombinant polynucleotides that has a desired property.

68. A recombinant polynucleotide obtained by the method of claim 1.

69. A vector comprising the polynucleotide of claim 68.

70. A cellular host transformed by the recombinant polynucleotide of claim 68.

71. A protein encoded by the recombinant polynucleotide of claim 68.

72. A library comprising the recombinant polynucleotide of claim 68.

73. A library comprising the vector of claim 69, the cellular host of claim 70 or the protein of claim 71.

74. A physical array in which the method of claim 1 can be performed.

75. A logical array that simulates the method of claim 1.

76. A logical array that simulates the physical array of claim 74.

77. The method of claim 1 or 53, wherein the steps occur in vivo.

78. A template-mediated, ligation-oriented method for nonrandomly shuffling polynucleotides, comprising:
a) hybridizing single stranded fragments of at least two homologous polynucleotides to one or more devised assembly templates until at least two of the fragments are adjacently hybridized, thereby forming at least one partially double-stranded polynucleotide, wherein at least one of said templates shares at least one zone of homology with said homologous polynucleotides;
b) treating said partially double-stranded polynucleotide to form at least one recombinant polynucleotide,
wherein said treating comprises, in any order, the following:
(i) ligating nicks, and
(ii) where necessary, any one of or any combination of the following gap filling techniques:
filling in gaps by further hybridizing said fragments to said templates to increase the number of fragments that are adjacently hybridized,
filling in short gaps by trimming any overhanging flaps of any partially hybridized fragments, and
filling in short gaps via polymerization.

79. A polynucleotide shuffling reaction mixture comprising:
single-stranded fragments of at least two homologous polynucleotides; and
at least one devised assembly template upon which at least two of the fragments can hybridize adjacently before any gap filling occurs.

80. An in vitro polynucleotide shuffling reaction mixture comprising:
solitary-stranded restriction fragments of at least two homologous polynucleotides; and
at least one devised assembly template upon which at least two of the restriction fragments can hybridize adjacently before any gap filling occurs, wherein the template is transient, solitary stranded and longer than all or substantially of the fragments.

81. A polynucleotide shuffling reaction mixture comprising:
free single-stranded fragments of at least two homologous polynucleotides;
at least one partially double-stranded polynucleotide comprising a strand of a devised assembly template and an opposite partial strand of hybridized fragments, wherein at least two of the hybridized fragments are adjacently hybridized before any gap filling occurs.

82. An in vitro polynucleotide shuffling reaction mixture comprising:
free solitary-stranded restriction fragments of at least two homologous polynucleotides;
at least one partially double-stranded polynucleotide comprising a strand of a devised solitary-stranded assembly template and an opposite partial strand of hybridized restriction fragments, wherein at least two of the hybridized restriction fragments are adjacently hybridized before any gap filling occurs and wherein the template strand is transient and longer than all or substantially of the free restriction fragments.

83. A composition of shuffled polynucleotides comprising:
at least one double-stranded recombinant polynucleotide comprising a strand of a devised assembly template and an opposite recombinant strand composed from previously free fragments, of at least two homologous polynucleotides, that hybridized to the template and were ligated, wherein at least two of the fragments that hybridized to the template hybridized adjacently before any gap filling occurred.

84. A composition of polynucleotides shuffled in vitro comprising
at least one double-stranded recombinant polynucleotide comprising a solitary-stranded devised assembly template and an opposite recombinant strand composed from previously free restriction fragments, of at least two homologous polynucleotides, that hybridized to the template and were ligated, wherein at least two of the fragments that hybridized to the template hybridized adjacently before any gap filling occurred, and wherein the template is transient and longer than all or substantially of the restriction fragments were before they hybridized to the template.

85. A composition of shuffled polynucleotides comprising:
at least one devised assembly template or at least one strand thereof; and
at least one recombinant polynucleotide comprising at least one recombinant strand composed from previously free fragments, of at least two homologous polynucleotides, that hybridized to the template and were ligated, wherein at least two of the fragments that hybridized to the template hybridized adjacently before any gap filling occurred, and wherein the recombinant strand and template separated after the recombinant strand was formed.

86. A composition of polynucleotides shuffled in vitro comprising:
at least one solitary-stranded devised assembly template;
at least one recombinant polynucleotide comprising at least one recombinant strand composed from previously free restriction fragments, of at least two homologous polynucleotides, that hybridized to the template and were ligated, wherein at least two of the fragments that hybridized to the template hybridized adjacently before any gap filling occurred, and wherein the recombinant strand and template separated after the recombinant strand was formed; and
wherein the template is longer than all or substantially all of the restriction fragments were before they hybridized to the template.

87. The method of claim 19, wherein the fragments at step (a) are about 15 residues in length.

88. The method of claim 22, wherein the fragments at step (a) are about 50-500 residues in length.

89. The method of claim 1, 53 or 78, wherein the at least two fragments that are adjacently hybridized are adjacently hybridized before any gap filling occurs.

90. The method of claim 1, 53, 63 or 78, wherein the at least two fragments that are adjacently hybridized are adjacently hybridized before any gap filling occurs and before any ligating occurs.
